(19) United States
(12) Patent Application Publication
(10) Pub. No.: US 2022/0118040 A1
(43) Pub. Date: Apr. 21, 2022

(54) COMPOUND ESSENTIAL OIL COMPOSITION AND USE THEREOF FOR IMPROVING COGNITION, SLEEP AND MOOD

(71) Applicant: Yueh-Yuan CHANG, Kaohsiung City (TW)

(72) Inventors: Yueh-Yuan CHANG, Kaohsiung City (TW); Shih-Guei FU, Kaohsiung City (TW); Ho-Cheng CHEN, Kaohsiung City (TW); Yu-Ting TU, Kaohsiung City (TW); Mei-Hsiu SHEN, Kaohsiung City (TW); Kuo-Wei LEE, Kaohsiung City (TW)

(21) Appl. No.: 17/075,868

(22) Filed: Oct. 21, 2020

(51) Int. Cl. A61K 36/537; A61K 36/53; A61K 31/352; A61K 36/534; A61K 36/185; A61P 25/28; A61P 25/20

(52) U.S. Cl. CPC A61K 36/537; A61K 36/53; A61K 31/352; A61P 25/20; A61K 36/185; A61P 25/28; A61K 36/534

(57) ABSTRACT

A compound essential oil composition includes a eucalyptol rosemary essential oil, a Lavandula angustifolia essential oil and a sweet marjoram essential oil and is suitable for use in daily life and outpatient treatment to improve cognition, sleep and mood. The physiological parameters and heart rate variability (HRV) of test subjects having sniffed the compound essential oil composition were measured.

### BACKGROUND OF THE INVENTION

### 1. Technical Field

The present invention relates to a compound essential oil composition and in particular relates to use thereof for improving cognition, sleep and mood.

### 2. Description of Related Art

Japan is the fastest aging country in the world. In recent years, Taiwan has followed Japan's footsteps and gradually become an aged society. In addition to those widely discussed chronic diseases in the aged population, mental health problems affect the quality of life of the elderly as well.

Please refer to Task Force of the European Society of Cardiology and the North American Society of Pacing and Electrophysiology. 1996 Mar. 1; 93(5):1043-65. and A Review of Heart Rate Variability and Its Association with Diseases. International Journal of Soft Computing and Engineering (IJSCE) ISSN: 2231-2307, Volume 2, Issue 3, July 2012. An autonomic nervous disorder refers to a condition in which a person's autonomic nervous system loses its balance with the dynamic regulation of the person's biological clock and physical and mental conditions, and in which the various organs regulated by the autonomic nervous system show their respective symptoms, resulting in other physical or mental disorders such as depression, anxiety and obesity, if not heart diseases or high blood pressure. The level of balance between sympathetic nervous system activity and parasympathetic nervous system activity can be known by the LF/HF ratio, whose recommended reference values are as follows: greater than 2.5 (relatively high), 1.2-2.5 (positive), 0.8-1.2 (balanced), 0.5-0.8 (relaxed) and less than 0.5 (relatively low). Stress can be said to be one of the most common causes of disease in today's society. People tend to suffer from physical or mental diseases of various degrees (including various common diseases related to the autonomic nervous system) when stress exceeds their adaptability.

While aromatherapy has been applied at many different levels, past research results have given rise to different opinions on the effects of essential oils on alleviating emotional disturbance, or have shown a lack of objective research evidence as the effects of essential oils were in most cases described by the test subjects subjectively.

A single essential oil of a concentration of 100%, e.g., a 100% lavender essential oil, is so pure that, for the sake of safety, it should not be brought into direct contact with the skin. Single essential oils must be diluted before use. The rules and methods of using single essential oils are far more complicated and difficult to understand than those of ordinary cosmetics, and most consumers are not familiar with the blending of essential oils. This explains why essential oils have yet to be widely used. A single or compound essential oil must be mixed with a carrier oil (also known as a base oil) before being applied in a spreading or massaging manner in order to be absorbed through the skin into the blood system and thus circulated through the body. A compound essential oil is a mixture of two or more pure single essential oils and can be diffused as an ambient scent.

The human body is a highly complex and sophisticated organism. There are more than 100 ingredients in essential oils, and the therapeutic properties of an essential oil are determined by its chemical composition. The special proportions of the ingredients of compound essential oils are not common knowledge but a key technology in essential oil preparation that cannot be obtained without long-term repeated trials. This is similar to traditional Chinese medicine prescriptions, whose fixed compositions include ingredients mixed at different weight ratios. For example, Li Hui mentions in Application of Siwutang in Gynecology of Traditional Chinese Medicine (Henan Traditional Chinese Medicine, 2012; 32(6):762) that siwutang (a decoction of four drugs) is so named because the four wu's (or the four substances) in siwutang are similar but still have their respective characteristics, each making its own contribution without conflicting with one another, the composition of siwutang including 2 qian of prepared rehmannia roots, 2 qian of Angelica sinensis, 2 qian of white peony roots and 2 qian of chuanxiong rhizome, wherein qian is a Chinese unit of measurement of weight.

In the Middle Ages, agarwood, bay laurel, rosemary, perilla sage, rose, frankincense, frangipani, lavender, nutmeg, absinthe, myrrh, elderberry and other spices were burnt in bonfire and were surprisingly effective in preventing the spread of plague. The famous physician Hua Tuo in the Eastern Han dynasty of China ground musk, clove and sandalwood into powder and hung the powder indoors to treat respiratory and upper digestive tract diseases.

There are many varieties of lavender, and the four major varieties that are used for the extraction of essential oils are Lavandula angustifolia, Lavandula burnatii, Lavandula latifolia and Lavandula stochas. Essential oils extracted from different varieties include different ingredients and are therefore suitable for treating different symptoms. The dosage of the Lavandula angustifolia essential oil is difficult to control. Many users reported that they could not sleep when using the Lavandula angustifolia essential oil in order to sleep better. Lavender essential oils serve as a sleep aid only when used at low concentrations; a strong lavender scent works contrarily and has a refreshing or invigorating effect. That is to say, Lavandula angustifolia and Lavandula burnatii are indeed capable of helping their users sleep but only have weakly soothing ingredients. Using lavender essential oils alone, therefore, still has its disadvantages. Chinese Patent No. 104800774B mentions a comparison of efficacy between four compound essential oil formulae and a single essential oil and states that the compound essential oils were more effective than the single essential oil and exhibited a synergistic effect. The different essential oil formulae, however, had different effects on people.

Sage essential oils contain thujone, which is a hypersensitive and highly toxic ingredient. Careless use of thujone may cause hallucinations, mental disorder or other intoxication symptoms in the human body. The clary sage essential oil, however, does not contain thujone.

The influence of aroma on implicit memory and human performance is discussed in Odors: Implicit Memory and Performance Effects, Chemical Senses, Oxford University Press, UK. According to the article, 108 subjects were randomly divided into three groups and entered three different spaces in which a jasmine essential oil, a lavender essential oil, and no essential oil were respectively diffused. The group corresponding to the use of no essential oil was the control group. The experiment lasted for 30 minutes, during which the subjects were required to complete arithmetic and spelling tests. While jasmine essential oils are generally believed to have an invigorating effect and lavender essential oils a calming effect, the results of the experiment show that the answer rate and correct answer rate of the lavender essential oil group were higher than those of the jasmine essential oil group. This is probably because the subjects who breathed in the lavender essential oil were brought into a favorable condition in which they were calmed down and relaxed both physically and mentally, under no stress, and hence able to think without distraction and analyze carefully while answering the questions. It can be known from the above that compound essential oils produce different effects on the human body as the ratios of their ingredients vary.

### BRIEF SUMMARY OF THE INVENTION

The problems to be solved by the present invention are as follows:

1. People nowadays tend to have emotional disturbance and sleep disorders due to busy work and a high level of stress in life.

2. People nowadays tend to have poor control of their emotional state and difficulty in concentration, and a lack of concentration leads to poor performance of the faculty of memory.

3. The main complaints of many patients indicate that they have such problems as depression, anxiety, poor overall cognitive performance and decreased short-term memory.

The present invention discloses a compound essential oil composition. Please refer to FIG. 1 for a flowchart of the compound essential oil composition of the present invention. The compound essential oil composition includes a eucalyptol rosemary essential oil, a Lavandula angustifolia essential oil and a sweet marjoram essential oil. The three ingredients are proportioned according to the following volume ratio: the eucalyptol rosemary essential oil:the Lavandula angustifolia essential oil:the sweet marjoram essential oil=2:1:1 (S101). The ingredients are then mixed (S102) to form the compound essential oil composition (S103).

The eucalyptol rosemary essential oil contains eucalyptol as the main ingredient. Eucalyptol is one of the inherent ingredients of lavender essential oils, e.g., the Lavandula latifolia essential oil. The other two ingredients of the compound essential oil composition of the present invention are the Lavandula angustifolia essential oil, which is one of the lavender essential oil varieties, and sweet marjoram essential oil. The three ingredients are mixed in different volume percentages to form the compound essential oil composition.

The present invention discloses another, or the second, compound essential oil composition. Please refer to FIG. 2 for a flowchart of the second compound essential oil composition of the present invention. The second compound essential oil composition includes a sweet orange essential oil, a peppermint essential oil, a clary sage essential oil and a sandalwood essential oil in addition to the ingredients of the previous compound essential oil composition. All the ingredients are proportioned according to the following volume ratio: 30-60 parts by volume of the eucalyptol rosemary essential oil, 25-40 parts by volume of the Lavandula angustifolia essential oil, 15-25 parts by volume of the sweet orange essential oil, 15-25 parts by volume of the peppermint essential oil, 5-12 parts by volume of the clary sage essential oil, 5-12 parts by volume of the sweet marjoram essential oil, and 5-12 parts by volume of the sandalwood essential oil (S201). The ingredients are then mixed (S202) to form the second compound essential oil composition (S203).

Apart from the eucalyptol rosemary essential oil, the Lavandula angustifolia essential oil and the sweet marjoram essential oil, the following four ingredients are added to the second compound essential oil composition: the clary sage essential oil, the sweet orange essential oil, the peppermint essential oil and the sandalwood essential oil. There are many varieties of sage, and one of them is “clary sage”. The pronunciation of clary is similar to “happy” in Chinese. The above seven ingredients are mixed in different volume percentages to form the second compound essential oil composition.

The present invention discloses yet another, or the third, compound essential oil composition, which includes a eucalyptol rosemary essential oil, a Lavandula angustifolia essential oil, a sweet orange essential oil, a peppermint essential oil, a clary sage essential oil, a sweet marjoram essential oil and a sandalwood essential oil. The seven ingredients are proportioned according to the following volume ratio: 40 parts by volume of the eucalyptol rosemary essential oil, 30 parts by volume of the Lavandula angustifolia essential oil, 20 parts by volume of the sweet orange essential oil, 20 parts by volume of the peppermint essential oil, 10 parts by volume of the clary sage essential oil, 10 parts by volume of the sweet marjoram essential oil, and 10 parts by volume of the sandalwood essential oil. The ingredients are then mixed to form the third compound essential oil composition.

The present invention discloses a compound essential oil composition and use thereof in a medicine for enhancing cognition. The use of the compound essential oil composition helps to enhance cognition. Cognitive enhancement can be assessed with the scales in the following common tests: the Mini-Mental State Examination (MMSE), the Cognitive Ability Screening Instrument (CASI) and the Stroop Color and Word Test (SCWT). The above tests can be used to score the following aspects of a subject: mental operation, attention, orientation, long-term memory, short-term memory, abstract thinking, graphic construction, language fluency, language skills, language acquisition-1, and language acquisition-all.

To begin with, a subject is required to fill in the Beck Depression Inventory-second edition (BDI-II) and the Beck Anxiety Inventory (BAI) to confirm their emotional state. If the scores of the two tests fall into the range of mild depression or mild anxiety, the subject will be invited to take part in the experiment. The subject's pre-experimental test items include: emotional state (BDI-II, BAI, blood pressure and heart rate variability analysis), cognitive functions (MMSE, cognitive function screening tools, Chinese Version Verbal Learning Test (CVVLT), Clinical Dementia Rating (CDR), computer-based cognitive training (CCT), and SCWT), sleep quality (the Pittsburgh Sleep Quality Index (PSQI)) and blood tests (cortisol and neurotrophic factor). The subject's intervention period lasts for six months, during which the subject receives aromatherapy. The subject's post-experimental test items are the same as the pre-experimental test items.

The experiment uses existing scales and research tools. The research tools include: BAI, BDI-II, PSQI, autonomic nervous system testing, CCT (cogniplus-HIBI)-based inhibitory control training, MMSE, CASI, CDR, SCWT, CVVLT and the Brain Derived Neurotrophic Factor (BDNF) test.

The experimental results are statistically analyzed using SPSS 16.0 to compare the subject's emotional state (BAI, BDI-II), sleep quality (PSQI) and cognitive functions (MMSE, CASI, CDR, CVVLT, SCWT, CCT and blood analysis results) before and after the intervention of aromatherapy.

It is generally known that CASI and SCWT are related to graphic construction, language skills, language acquisition-1, language acquisition-all and cognitive functions, all of which are in turn related to the present invention.

Referring to FIG. 3, MMSE is conducted for research in accordance with the questionnaire on page 499 of the Journal of Neurology, Neurosurgery, and Psychiatry. 1984; 47:496-499. MMSE is an assessment tool designed for the cognitive functions. The examination includes a total of eleven questions related to such cognitive functions as those regarding time, orientation, attention, arithmetic ability, language skills (including reading, writing, naming, comprehension and operation), immediate memory, short-term memory, and visual-spatial ability. Each question is assigned a different score, and the total score of the examination is 30. The higher the total score of the examination, the better the performance.

CASI is conducted for research in accordance with the questionnaire in International Psychogeriatrics, Vol. 6, No. 1, 1994, with a score ranging from 0 to 100. The questionnaire can be used to assess attention, direction, short-term memory, long-term memory, language skills, visual structure, fluency in list generation, abstraction and judgment.

For SCWT, please refer to Stroop Effect in the Bilingual Vocabulary, Academic Term and Dictionary Information Network of the National Academy for Educational Research. Generally, this effect shows the subject's cognitive process. When a new source of stimulus appears, and if the characteristics of this source of stimulus are similar to or consistent with the stimulus received from an existing source of stimulus, the cognition of the new stimulus will be accelerated. On the contrary, if the characteristics of the new source of stimulus are different from those of the existing ones, the subject's cognition will be disturbed such that it takes a longer time for the subject to respond to the new source of stimulus than to the existing ones.

Autonomic nervous system testing includes tests on the sudomotor function, the vagus nerve, and the adrenergic function. The testing methods include: the subject taking deep breaths in a unit time, performing the Valsalva maneuver (VM), receiving the head-up tilt tests, and receiving static blood pressure and ECG recording for 5-10 minutes for heart rate variability analysis. The data collected during the measurement period are as follows: heart rate response to deep breathing (HRDB), the Valsalva ratio, beat-to-beat blood pressure measurements during the Valsalva maneuver, orthostatic BP and HR tested with a tilted bed, and baroreflex sensitivity (BRS).

The present invention also discloses use of the foregoing compound essential oil composition in a medicine for improving mood. The use of the compound essential oil composition helps to improve mood. Mood improvement can be assessed with the following items: BAI, physical symptoms, cognitive beliefs, and the Beck Depression Inventory (BDI). The present invention further discloses use of the compound essential oil composition in a medicine for improving sleep quality. The use of the compound essential oil composition helps to improve sleep quality. Sleep quality can be assessed with the following items: sleep quality index, subjective sleep quality, sleep delay, sleep efficiency, use of medication, sleep disturbance and daytime dysfunction.

The roles of the sympathetic nerves and of the parasympathetic nerves are briefly stated as follows. A person's autonomic nervous system changes dynamically with the person's physical and mental conditions and biological clock. The sympathetic nerves mainly control excitement and metabolism, whereas the parasympathetic nerves mainly control relaxation and storage. With the sympathetic nerves and the parasympathetic nerves working properly with each other (which is analogous to the operations of the accelerator and the brake of a vehicle), the human body can operate smoothly and have a long service life.

Clinical application of heart rate variability (HRV) measurement complies with the international standard for the “measurement, physiological significance and clinical application of heart rate variability”. Heart rate variability is used to assess the relationship between the autonomic nervous system and various physical and mental diseases. Heart rate variability analysis is an analytical method that measures changes in continuous heart rate. A three-lead electrocardiogram is continuously recorded so that the subject's heartbeat can be calculated, and a photoplethysmography (PPG) instrument is used to capture the signal from the subject's finger to measure blood pressure (BP).

BAI and BDI are briefly described as follows. BAI, which was created by Professor Aaron T. Beck and other psychiatrists in the United States in 1988, uses a 21-question inventory to assess the level of anxiety of a subject. BAI is suitable for teenagers and adults aged 17 or over. Each question has 4 choices: 0 (not at all) to 3 (severely). The subject is required to choose the degree of distress experienced from each symptom based on what happened in the past week, and the test result is obtained by calculating the sum of all the scale values. The higher the sum, the more serious the anxiety. BDI, on the other hand, is a scale commonly used to measure depression. BDI was developed by psychologist Aaron T. Beck and others in 1961 and was later modified into the BDI-II commonly used today. The reliability coefficient of BDI ranges from 0.70 to 0.93. BDI-II was adopted in the experiment for the present invention.

As mentioned above, the present invention discloses use of the foregoing compound essential oil composition in a medicine for improving sleep quality. The use of the compound essential oil composition helps to improve sleep quality.

PSQI assesses sleep quality according to the following items: sleep quality index, subjective sleep quality, sleep delay, sleep efficiency, use of medication, sleep disturbance and daytime dysfunction. The score for each of the above seven aspects ranges from 0 to 3, so the total score ranges from 0 to 21. A PSQI score greater than 5 indicates that the subject has sleep disturbance. The higher the score, the worse the sleep quality.

The present invention discloses use of the foregoing compound essential oil composition in preparing a medicine for enhancing cognition, a medicine for improving mood, or a medicine for enhancing sleep quality.

The compound essential oil composition of the present invention can be used to achieve not only one, but also all, of cognition enhancement, mood improvement and sleep quality enhancement. Cognition enhancement and mood improvement belong to the psychological level, and sleep quality enhancement the physical level. By using the compound essential oil composition of the present invention, both rather than only one of the physical and the psychological levels can be taken care of. It is therefore obvious that the compound essential oil composition of the present invention works better than other compound essential oils.

The foregoing medicine is administrated by sniffing.

Aromatherapy, etymologically derived from the French terms for “aroma” and “therapy”, uses essential oils extracted from aromatic plants by distillation, and the essential oils can be administrated by direct inhalation, bathing, massaging, etc. Aromatherapy is carried out mainly in two ways: through the sense of smell (i.e., inhalation through the nose) and/or direct contact (i.e., absorption through the skin). The foregoing medicine is administrated by sniffing.

The foregoing medicine is administered twice a day for 15-60 minutes each time. The sniffing time is preferably 20-40 minutes and more preferably 30 minutes.

The compound essential oil composition of the present invention can be used as a massage oil after being added with a base oil. The most effective base oil is pure vegetable oil, which serves the purposes of not only dilution, buffering and assisting in the absorption and penetration of pure essential oil, but also desensitization.

In addition to the compound essential oil composition, an antibacterial ingredient may be added to make an antibacterial preparation for external use.

In addition to the compound essential oil composition, a cleaning ingredient may be added to make a cleaning agent for cleaning the human body or the environment.

In addition to the compound essential oil composition, a skincare ingredient may be added to make cosmetics for body care or for the improvement of body and mind.

In addition to the compound essential oil composition, an antibacterial ingredient, a cleaning ingredient, a skincare ingredient or a combination thereof may be added to make a multifunctional product that serves antibacterial, cleaning and skincare purposes at the same time.

The compound essential oil composition is prepared by a solvent extraction method, a distillation method, a cold pressing method, a pressing method or a carbon dioxide extraction method.

Preparations of the compound essential oil composition may be in the form of an aerosol, a spray, a fragrance product for use with diffusers or a nasal inhalant.

The compound essential oil composition is pure essential oil of organic sources.

Essential oils can be divided into four grades. The first grade is pure essential oil distilled at an appropriate temperature from cultivated plants of natural organic sources. Organic sources refer to natural raw materials that are produced in certified organic farms, that do not go through animal testing, that are not sterilized with radioactive rays, and that are not genetically modified (non-GMO). Essential oil products comply with the highest standards and are certified by gas chromatography-mass spectrometry (GC-MS) tests in accordance with the certification standards of the International Organization for Standardization (ISO).

The main effects of the present invention are:

1. A soothing effect can be produced on subjects with emotional disturbance and sleep disorders.

2. By improving the emotional state, a subject's attention can be regulated to improve concentration and thereby enhance the performance of the faculty of memory.

3. Overall cognitive performance and short-term memory are significantly improved as well as such main complaints as depression and anxiety.

4. Not only one, but also all, of cognition enhancement, mood improvement and sleep quality enhancement can be achieved. By using the compound essential oil composition of the present invention, both the physical and the psychological levels are taken care of.

### BRIEF DESCRIPTION OF THE SEVERAL VIEWS OF THE DRAWINGS

FIG. 1 is a flowchart of a compound essential oil composition of the present invention.

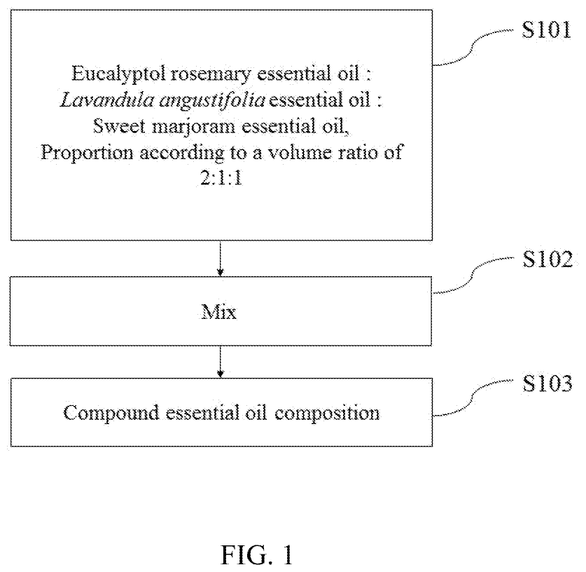

FIG. 2 is a flowchart of another compound essential oil composition of the present invention.

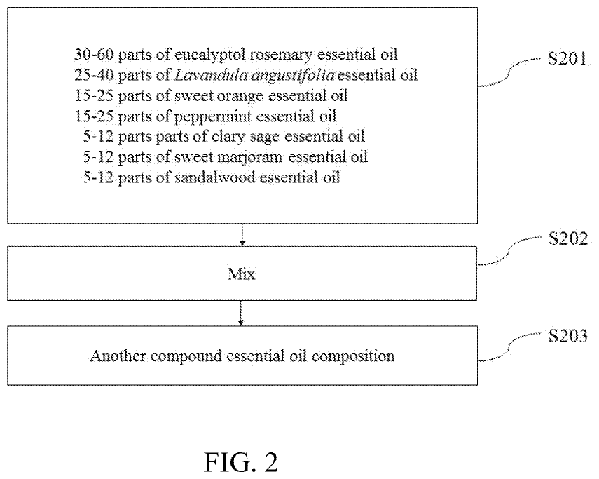

FIG. 3 is a flowchart of Mini-Mental State Examination (MMSE).

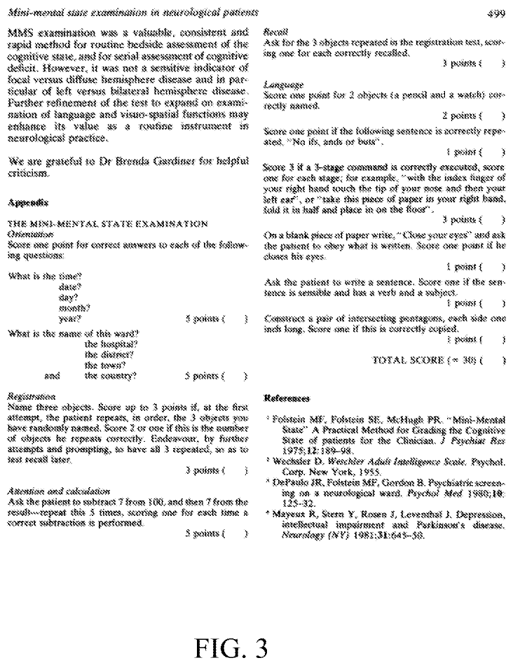

### DETAILED DESCRIPTION

### Embodiment 1

14 liters of a compound essential oil composition of the present invention was prepared from organic ingredients extracted from natural plants by way of example. The compound essential oil composition contained a eucalyptol rosemary essential oil, a Lavandula angustifolia essential oil, a sweet orange essential oil, a peppermint essential oil, a clary sage essential oil, a sweet marjoram essential oil and a sandalwood essential oil. The above ingredients were proportioned according to the following volume ratio:

| | | |
|---|---|---|
| eucalyptol rosemary essential oil | 4 | liters |
| Lavandula angustifolia essential oil | 3 | liters |
| sweet orange essential oil | 2 | liters |
| peppermint essential oil | 2 | liters |
| clary sage essential oil | 1 | liter |
| sweet marjoram essential oil | 1 | liter |
| sandalwood essential oil | 1 | liter. |

The preparation method is as follows: The eucalyptol rosemary essential oil, the Lavandula angustifolia essential oil, the sweet orange essential oil, the peppermint essential oil, the clary sage essential oil, the sweet marjoram essential oil and the sandalwood essential oil were successively added, at room temperature, into a mixer. The mixer was subsequently sealed, and the above seven essential oils were mechanically mixed at a low rotation speed (in a range of 20-100 rpm).

The compound essential oil composition obtained was ready for use with a constant-temperature diffuser, with the temperature of the diffuser controlled at 70° C. so as not to damage the structure of the composition. 3-5 drops of the composition, for example, were applied to the diffuser to enable aromatherapy by sniffing.

A preliminary test was conducted on the effects of aromatherapy in treating emotional disturbance.

Test subjects: A total of 31 outpatients (25 females and 6 males) at the Division of Neurology of Kaohsiung Chang Gung Memorial Hospital were chosen as the test subjects. The test subjects were 20-70 years old, with an average age of 51.81±11.18 years and an average education level of 12.90±3.37 years.

Test conditions: The test subjects were those who had memory complaints, suffered from emotional disturbance, but did not have obvious degeneration, brain injury or epilepsy, as diagnosed by physicians at the Division of Neurology of Kaohsiung Chang Gung Memorial Hospital. Moreover, the subjects had normal olfactory functions, a BAI score of 10-18 (mild to moderate anxiety) and a BDI-II score of 14-19 (mild depression). As used herein, the term “before intervention” means “before treatment with the compound essential oil composition”, and the term “after intervention” means “after treatment with the compound essential oil composition”.

Usage: Twice a day (in the morning before 12 a.m. and in the afternoon before 8 p.m.), each subject added six drops of the essential oil composition into a diffuser and sniffed for 30 minutes. Also, each subject wore an essential oil necklace into which the compound essential oil composition of the present invention had been added in drops. The subjects were required to keep daily records of their sniffing sessions and the wearing of necklaces. During the course of the research, the subjects received the compound essential oil composition only by sniffing from their diffusers and essential oil necklaces; the compound essential oil composition was never administered by massaging or other methods.

Judgment of results: It was determined whether the subjects showed any changes in cognitive functions and emotional reliefs after using the compound essential oil composition, by sniffing, for six months. Statistical analysis was performed with SPSS 16.0. The subjects' emotional states, sleep quality and cognitive functions were analyzed before and after the intervention.

Experimental Results

CASI, graphic construction, language acquisition-1, language acquisition-all and SCWT are related to people's cognition. According to the results of the preliminary test as shown in Table 1, the subjects showed significant improvement in such cognitive functions as memory (p=0.03<0.05), concentration (p=0.000<0.001) and execution ability (p=0.008<0.01) after using the compound essential oil composition of the present invention. The number in each item field of Table 1 represents the total score of the corresponding assessment.

TABLE 1
Assessment of cognitive functions

| | Before | After | |
|---|---|---|---|
| | intervention | intervention | |
| Item | (n = 36) | (n = 31) | p |
| MMSE (30) | 27.97 ± 1.96 | 28.52 ± 1.73 | 0.081 |
| CASI (100) | 93.36 ± 5.08 | 94.73 ± 5.02 | 0.042* |
| Mental operation (10) | 8.81 ± 1.51 | 8.84 ± 1.37 | 0.897 |
| Attention (8) | 7.32 ± 0.91 | 7.58 ± 0.72 | 0.088 |
| Orientation (18) | 17.87 ± 0.43 | 17.71 ± 0.69 | 0.282 |
| Long-term memory (10) | 10.00 ± 0 | 10.00 ± 0 | |
| Short-term memory (12) | 10.75 ± 1.61 | 11.22 ± 1.43 | 0.074 |
| Abstract thinking (12) | 10.74 ± 1.24 | 10.71 ± 0.90 | 0.839 |
| Graphic construction (10) | 9.58 ± 0.76 | 9.84 ± 0.44 | 0.030* |
| Language fluency (10) | 8.58 ± 1.86 | 8.84 ± 1.86 | 0.413 |
| Language skills (10) | 9.47 ± 1.59 | 9.73 ± 0.63 | 0.363 |
| Language acquisition-1 (9) | 5 ± 2.28 | 6.32 ± 1.51 | 0.000*** |
| Language acquisition-all | 26.03 ± 8.50 | 29.90 ± 4.07 | 0.003** |
| (36) | | | |
| SCWT (63) | 45.58 ± 15.27 | 50.65 ± 11.09 | 0.008** |

Note:
MMSE stands for Mini-Mental State Examination, and
CASI stands for Cognitive Ability Screening Instrument.

As for the emotional state, BAI and BDI were used for assessment. As shown in Table 2, the values corresponding to anxiety and depression decreased significantly (e.g., p=0.000<0.001) after the use of the compound essential oil composition of the present invention. According to the data, the subjects' anxiety and depression were improved, physical symptoms associated with anxiety and depression reduced, and negative cognitive beliefs lessened.

TABLE 2
Assessment of emotional state

| | Before | After | |
|---|---|---|---|
| | intervention | intervention | |
| Item | (n = 36) | (n = 31) | p |
| BAI | 17.94 ± 12.81 | 6.32 ± 6.23 | 0.000*** |
| Physical symptoms | 7.39 ± 5.99 | 2.39 ± 2.91 | 0.000*** |
| Cognitive beliefs | 10.32 ± 7.19 | 3.94 ± 3.94 | 0.000*** |
| BDI | 18.71 ± 12.08 | 9.90 ± 11.01 | 0.000*** |
| Physical symptoms | 18.71 ± 12.08 | 9.90 ± 11.01 | 0.000*** |
| Cognitive beliefs | 5.71 ± 3.16 | 2.19 ± 1.94 | 0.001** |

As for sleep quality, a comparison of the indexes before and after using the compound essential oil composition of the present invention shows that, judging from the p-value, both sleep delay (p=0.006<0.01) and the use of sleep aids (p=0.027<0.05) were reduced. According to the data, sleep quality was significantly improved.

TABLE 3
Assessment of sleep quality

| | Before | After | |
|---|---|---|---|
| | intervention | intervention | |
| Item | (n = 36) | (n = 31) | p |
| Sleep quality index | 12.87 ± 4.39 | 9.38 ± 4.48 | 0.000*** |
| Subjective sleep quality | 2.91 ± 0.75 | 1.45 ± 0.72 | 0.000*** |
| Sleep delay | 2.10 ± 0.87 | 1.52 ± 1.23 | 0.006** |
| Sleep efficiency | 1.39 ± 1.26 | 1.32 ± 1.35 | 0.606 |
| Use of medication | 1.61 ± 1.48 | 1.10 ± 1.38 | 0.027* |
| Sleep disturbance | 1.85 ± 0.69 | 1.16 ± 0.52 | 0.000*** |
| Daytime dysfunction | 1.58 ± 0.89 | 0.84 ± 0.82 | 0.000*** |

As to the autonomic nervous system, which maintains the essential functions of life, the subjects' LF and HF did not change significantly after the intervention of the compound essential oil composition of the present invention. Moreover, once the factor of “whether the subjects kept their daily records continuously and diligently” was taken into account, a research observation of the subjects' LF/HF ratios shows that those who “monitored their own states continuously” improved significantly in terms of LF/HF (p=0.009).

LF is a sympathetic nerve-related composite activity index corresponding to sympathetic nervous system activity and parasympathetic nervous system activity, and HF is an index of parasympathetic nerve system activity. The LF/HF ratio is an index of the level of balance between sympathetic nervous system activity and parasympathetic nervous system activity and can be quantitatively derived from the high- and low-frequency bands of heart rate variability as a measurement of the interaction between the sympathetic nerves and the parasympathetic nerves. An exceedingly low value of heart rate variability is an important index of autonomic nerve-related diseases.

According to the data in Table 4 and the recommended reference values of the LF/HF ratio, which indicates the level of balance between sympathetic nervous system activity and parasympathetic nervous system activity, the ratio before the intervention of the compound essential oil composition of the present invention was less than 0.5 (relatively low) and ranged from −2.17 to 0.55, while the ratio after the intervention was increased by about 1.2 and ranged from −0.56 to 1.36, which range is close to the range of 0.5 to 0.8 (relaxed).

TABLE 4
Assessment of autonomic nervous system

| Item | Before intervention | After intervention | p |
|---|---|---|---|
| LF/HF | −0.81 ± 1.36 | 0.40 ± 0.96 | 0.009** |
| (D-ratio) | | | |

The experimental results show that the compound essential oil composition of the present invention had a soothing effect on subjects suffering from emotional disturbance and sleep disorders. The improvement of the emotional state helped regulate the subjects' attention, and the improvement of concentration enhanced the performance of the faculty of memory. In addition to significant improvement in such main complaints as depression and anxiety, overall cognitive performance and short-term memory were also significantly improved.

### Embodiment 2

10 liters of a compound essential oil composition of the present invention was prepared from organic ingredients extracted from natural plants by way of example. The compound essential oil composition contained a eucalyptol rosemary essential oil, a Lavandula angustifolia essential oil, a sweet orange essential oil, a peppermint essential oil, a clary sage essential oil, a sweet marjoram essential oil and a sandalwood essential oil. The above ingredients were proportioned according to the following volume ratio:

| | | |
|---|---|---|
| eucalyptol rosemary essential oil | 3.0 | liters |
| Lavandula angustifolia essential oil | 2.5 | liters |
| sweet orange essential oil | 1.5 | liters |
| peppermint essential oil | 1.5 | liters |
| clary sage essential oil | 0.5 | liter |
| sweet marjoram essential oil | 0.5 | liter |
| sandalwood essential oil | 0.5 | liter. |

### Embodiment 3

100 liters of a compound essential oil composition of the present invention was prepared from organic ingredients extracted from natural plants by way of example. The compound essential oil composition contained a eucalyptol rosemary essential oil, a Lavandula angustifolia essential oil and a sweet marjoram essential oil. The above ingredients were proportioned according to the following volume ratio:

| | | |
|---|---|---|
| eucalyptol rosemary essential oil | 50 | liters |
| Lavandula angustifolia essential oil | 25 | liters |
| sweet marjoram essential oil | 25 | liters. |

The preparation method is as follows: The eucalyptol rosemary essential oil, the Lavandula angustifolia essential oil and the sweet marjoram essential oil were successively added, at room temperature, into a mixer. The mixer was subsequently sealed, and the above three essential oils were mechanically mixed at a low rotation speed (in a range of 20-100 rpm).

The compound essential oil composition obtained was ready for use with a constant-temperature diffuser, with the temperature of the diffuser controlled at 60-75° C. 3-5 drops of the composition, for example, were applied to the diffuser to enable aromatherapy by sniffing.

The embodiments provided herein are merely some preferred ones of the present invention that serve to illustrate the technical means with which to solve the problems stated above. Those embodiments are not intended to limit the scope of the present invention. Any equivalent changes or modifications that match the literal scope of, or are made according to, the claims of the present invention shall fall within the scope of the present invention.

What is claimed is:

1. A compound essential oil composition, comprising: a eucalyptol rosemary essential oil, a Lavandula angustifolia essential oil and a sweet marjoram essential oil, wherein the eucalyptol rosemary essential oil, the Lavandula angustifolia essential oil and the sweet marjoram essential oil are proportioned according to the following volume ratio and are mixed to form the compound essential oil composition:the eucalyptol rosemary essential oil:the Lavandula angustifolia essential oil:the sweet marjoram essential oil=2:1:1.

2. The compound essential oil composition according to claim 1, further comprising a sweet orange essential oil, a peppermint essential oil, a clary sage essential oil and a sandalwood essential oil, wherein the eucalyptol rosemary essential oil, the Lavandula angustifolia essential oil, the sweet orange essential oil, the peppermint essential oil, the clary sage essential oil, the sweet marjoram essential oil and the sandalwood essential oil ingredients are proportioned according to the following volume ratio and are mixed to form the compound essential oil composition: 30-60 parts by volume of the eucalyptol rosemary essential oil, 25-40 parts by volume of the Lavandula angustifolia essential oil, 15-25 parts by volume of the sweet orange essential oil, 15-25 parts by volume of the peppermint essential oil, 5-12 parts by volume of the clary sage essential oil, 5-12 parts by volume of the sweet marjoram essential oil, and 5-12 parts by volume of the sandalwood essential oil.

3. The compound essential oil composition according to claim 2, wherein the eucalyptol rosemary essential oil, the Lavandula angustifolia essential oil, the sweet orange essential oil, the peppermint essential oil, the clary sage essential oil, the sweet marjoram essential oil and the sandalwood essential oil are proportioned according to the following volume ratio and are mixed to form the compound essential oil composition: 40 parts by volume of the eucalyptol rosemary essential oil, 30 parts by volume of the Lavandula angustifolia essential oil, 20 parts by volume of the sweet orange essential oil, 20 parts by volume of the peppermint essential oil, 10 parts by volume of the clary sage essential oil, 10 parts by volume of the sweet marjoram essential oil, and 10 parts by volume of the sandalwood essential oil.

4. Use of the compound essential oil composition according to claim 1 in preparing a medicine for enhancing cognition.

5. Use of the compound essential oil composition according to claim 1 in preparing a medicine for improving mood.

6. Use of the compound essential oil composition according to claim 1 in preparing a medicine for enhancing sleep quality.

7. Use of the compound essential oil composition according to claim 1 in preparing a medicine for enhancing cognition, improving mood, and enhancing sleep quality.

8. The compound essential oil composition according to claim 1, wherein the compound essential oil composition is administered by sniffing twice a day for 15-45 minutes each time.
